(19) United States
(12) Patent Application Publication
(10) Pub. No.: US 2023/0140095 A1
(43) Pub. Date: May 4, 2023

(54) DETACHMENT MECHANISM WITH A TAB FOR DELIVERING INTRAVASCULAR IMPLANTS

(71) Applicant: SPARTAN MICRO, INC., Costa Mesa, CA (US)

(72) Inventors: Yoshitaka Katayama, Costa Mesa, CA (US); Michael D. Martel, Costa Mesa, CA (US); Quan Ngo, Costa Mesa, CA (US); Eric P. Stoppenhagen, Costa Mesa, CA (US)

(21) Appl. No.: 17/452,907

(22) Filed: Oct. 29, 2021

(51) Int. Cl. A61B 17/12

(52) U.S. Cl. CPC A61B 17/1214; A61B 17/12113; A61B 2017/12054

(57) ABSTRACT

Systems and methods are provided for delivering and mechanically detaching embolic coils. The systems disclosed herein comprise a mechanical detachment mechanism to intravascularly release an embolic coil. The methods disclosed herein comprise triggering mechanisms to detach embolic coils.

### BACKGROUND

A number of vascular disorders are treated by an intravascular delivery of an implant that is either positioned or deployed within a vessel of a body of an individual. For example, an intravascular stent used for treating peripheral artery disease may be deployed in a stenotic region of a blood vessel in order to improve blood flow past the stenosis in the vessel. For further example, an embolic coil may be placed or deployed within an intracerebral aneurysm in order to occlude the aneurysm thus preventing blood flow into the aneurysm and thus preventing a rupture of the aneurysm.

### SUMMARY

Described herein are systems and methods for delivering an intravascular implant. The systems and methods described herein use an intravascular approach for delivering an implant into the intravascular system of a patient. In some embodiments of the systems and methods described herein, the systems and methods comprise a mechanical detachment system that is configured to deploy an intravascular implant, such as an embolic coil, at a target location within the vascular system of a patient when a user manually deploys the implant.

In some embodiments of the systems and methods described herein, the systems and methods are used for the delivery of an embolic coil to an intracranial aneurysm and are configured to provide manually triggered deployment of an embolic coil within the intracranial aneurysm.

The systems and methods described herein improve upon traditional implant detachment systems, such as, for example, embolic coil detachment systems, in a number of ways:

### Prevention of Undesired Thrombotic Events

One example of how the systems and methods described herein improve on traditional systems and methods for delivering embolic coils is by preventing undesired thrombotic events.

Many traditional systems and methods for delivering embolic coils to cerebral aneurysms employ electrolytic detachment mechanisms, which have been shown to cause generation of gas bubbles at the detachment zone. The formation of gas bubbles intravascularly leads to the formation of blood clots, which may lead to thromboembolic complications. Furthermore, if the clot remains attached to a micro-catheter tip or to the end of an embolic coil, there is a risk that the clot will grow in size and/or embolize during repeated embolic coil detachment procedures. This presents an increased risk of the generation of blood clots that can travel to small vessels and occlude these vessels, leading to anoxic injury.

### Decrease in Detachment Time

Another example of how the systems and methods described herein improve on traditional systems and methods of delivering embolic coils is by decreasing the time to detach the total number of embolic coils.

The systems and methods described herein take significantly less time to detach and deploy an embolic coil as compared to traditional electrolytic systems. The systems and methods described herein comprise mechanical components that actuate rapid deployment whereas electrolytic systems require time to heat an embolic coil system in order to detach and deploy a coil. As, in most cases, delivery of multiple embolic coils into one cerebral aneurysm is often necessary, the reduction in procedure time by the systems and methods described herein presents a significant advantage.

### Prevention of Detachment Failure

Yet another example of how the systems and methods described herein improve on traditional systems and methods of delivering embolic coils is by preventing a failed detachment of an embolic coil.

Traditional electrolytic detachment systems and methods have been shown to have a significant detachment failure rate. Detachment failure may occur due to electrical equipment failure and/or failure to properly induce a current through the device and the patient. Because the systems and methods described herein employ mechanical components rather than electrical components, the failure rate is significantly lower than that of the traditional electrolytic deployment systems.

Described herein is an embolic coil delivery system for delivering and deploying an embolic coil at an aneurysm comprising:
i. an embolic coil with an anchoring element disposed at a proximal end of the embolic coil;
ii. a conduit having a deployment location from which the embolic coil is deployed, and a first radiopaque marker:
iii. a detachment system configured to fit within the conduit and to be easily advanced and withdrawn within the conduit, the detachment system comprising:
   a. a detachment mechanism comprising:
      1) a housing that defines a tab with a first end coupled to a proximal end of the housing and a free end that extends toward a distal end of the housing, the tab comprising a shape memory material wherein the tab is configured to move from a first position to a second position, wherein when the tab is in the first position, the fee end of the tab is deflected toward an interior of the housing, wherein the tab comprises a passthrough clearance aperture; and
      2) a primary member configured and positioned to engage with the tab (extends through the passthrough clearance aperture) so that the tab is in the first position when engaged with the primary member and is moved to the second position by the memory material when the primary member is no longer engaged with the tab,
      3) wherein the anchoring element of the embolic coil is configured and positioned to engage with the tab (and extends through the passthrough clearance aperture of the tab) in the first position so that the embolic coil is coupled to the detachment system when the tab is in the first position, and wherein the anchoring mechanism is configured and positioned to not engage with the tab in the second position so that the embolic coil is deployed when the tab is in the second position;
      4) a radiopaque marker coupler; and
   b. a second radiopaque marker that is mechanically coupled with the radiopaque marker coupler and is positioned to align with the first radiopaque marker when the detachment mechanism is positioned at the deployment location.

In some embodiments of the delivery system, the detachment system comprises a flexible tube that surrounds the detachment system and fixedly couples the radiopaque marker coupler and the radiopaque marker. In some embodiments of the delivery system, the first radiopaque marker partially surrounds the conduit so that when the detachment mechanism is advanced within the conduit and the first radiopaque marker aligns with the second radiopaque marker, the second radiopaque marker is radiographically visible under fluoroscopy. In some embodiments of the delivery system, the tab comprises a shape memory metal material. In some embodiments of the delivery system, the detachment mechanism further comprises a primary member that detachably couples with the tab so that when the primary member and the tab are coupled, the tab is in the first position and when the primary member and the tab are decoupled, the tab moves to the second position. In some embodiments of the delivery system, the tab moves to the second position when the primary member is drawn away from the tab. In some embodiments of the delivery system, the detachment system includes a segment that is configured to manually detach from the detachment system, and wherein the primary member is coupled to the segment so that when the segment is manually detached and withdrawn away from the detachment system, the primary member is drawn away from the tab so that the tab moves to the second position and deploys the coil. In some embodiments of the delivery system, the segment comprises oblong cuts around its outer diameter that are configured to fracture the segment when a bending force is applied to the segment.

Also described herein is a method for deploying an embolic coil in an intracranial aneurysm comprising: directing a conduit through one or more blood vessels of the patient to the aneurysm, the conduit comprising a first radiopaque marker and a deployment location; advancing a detachment system through the conduit while the conduit is within the blood vessel, the detachment system comprising a radiopaque marker coupler, a second radiopaque marker, and a detachment mechanism comprising a tab having a first position and a second position; deploying the embolic coil within the aneurysm using the detachment system; wherein the radiopaque marker coupler and the second radiopaque marker couple mechanically; wherein when the detachment mechanism is positioned at the deployment location, the first radiopaque marker and the second radiopaque marker align; wherein the embolic coil is coupled to an anchoring element; wherein when the tab is in the first position, the anchoring element engages the tab thus coupling the embolic coil to the detachment system; and wherein the anchoring element does not engage the tab in the second position thus decoupling the embolic coil from the detachment system and thus deploying the embolic coil in the intracranial aneurysm. In some embodiments of the method, the detachment system comprises a flexible tube that surrounds the detachment system and fixedly couples the radiopaque marker coupler and the radiopaque marker. In some embodiments of the method, the first radiopaque marker partially surrounds the conduit so that when the detachment system is advanced within the conduit and the first radiopaque marker aligns with the second radiopaque marker, the second radiopaque marker is radiographically visible. In some embodiments of the method, the tab comprises a memory metal material. In some embodiments of the method, the detachment mechanism further comprises a primary member that detachably couples with the tab so that when the primary member and the tab are coupled, the tab is in the first position, and when the primary member and the tab are decoupled, the tab moves to the second position. In some embodiments of the method, the step of deploying comprises decoupling the primary member from the tab by drawing the primary member away from the tab. In some embodiments of the method, the conduit includes a segment that is configured to manually detach from the conduit, and wherein the primary member is coupled to the segment so that when the segment is manually detached and withdrawn away from the conduit, the primary member is drawn away from the tab so that the tab moves to the second position and deploys the embolic coil. In some embodiments of the method, the segment comprises oblong cuts around its outer diameter that are configured to fracture the segment when a bending force is applied to the segment.

### BRIEF DESCRIPTION OF THE DRAWINGS

The novel features of the subject matter disclosed herein are set forth with particularity in the appended claims. A better understanding of the features and advantages of the subj ect matter disclosed herein will be obtained by reference to the following detailed description that sets forth illustrative embodiments, in which the principles of the subject matter disclosed herein are utilized, and the accompanying drawings of which:

FIG. 1 shows an illustration of the anatomical path of travel for a delivery system according to one embodiment of the present disclosure.

FIG. 2 shows an illustration of an exemplary embodiment of a delivery system for delivering and deploying an intravascular implant such as an embolic coil.

FIG. 3 shows an illustration of an exemplary embodiment of a detachment system that comprises an embolic coil that is detachably coupled to the detachment system of FIG. 2.

FIG. 4 shows a perspective view of the detachment system of FIG. 3 in a locked configuration.

FIG. 5 shows a perspective view of an embodiment of a distal end of the detachment system of FIG. 3 in an unlocked configuration as the embolic coil is deployed from the detachment system.

FIG. 6 shows an illustration of an exemplary embodiment of a detachment system with a cover covering the detachment system.

FIG. 7 shows an exemplary illustration of an embodiment of an expansion tube of a delivery system.

### DETAILED DESCRIPTION

Described herein are systems and methods for delivering and deploying an intravascular implant to an intravascular target such as, for example, delivering one or more embolic coils to an intracranial aneurysm and deploying the one or more embolic coils within the aneurysm.

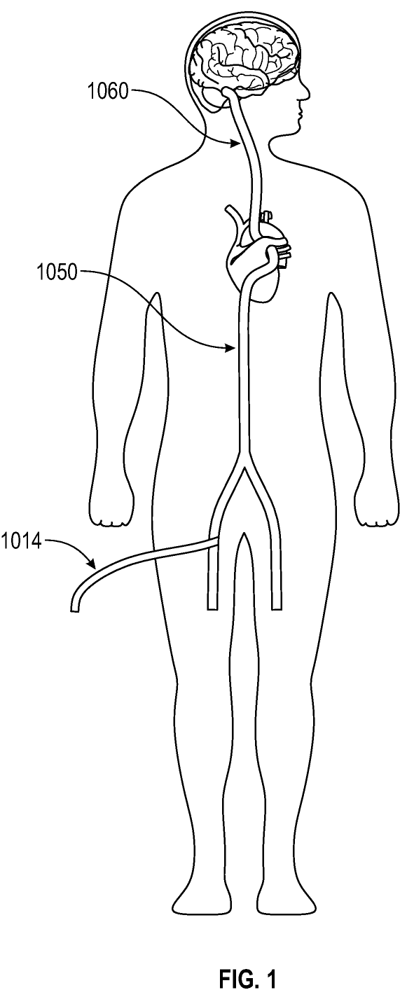

A delivery system as described herein comprises a conduit such as a traditional catheter or micro-catheter and a detachment system that is configured to be slideably advanced within the catheter. The catheter of the delivery system described herein is configured to be advanced through a blood vessel of a patient to a target location. For example, FIG. 1 shows an illustration of the anatomical path of travel of a catheter 1014. The catheter 1014 may be inserted into a femoral artery of a patient (using, for example, Seldinger technique) and advanced up through the aorta 1050 of the patient. From there, the catheter 1014 may be advanced up through a carotid artery 1060 to an intracranial target location such as an intracranial aneurysm where an intravascular implant may be deployed in order to, for example, occlude the aneurysm thus preventing aneurysm rupture. The conduit of the delivery system is thus configured to deliver the detachment system described herein to a target location. In some embodiments, the delivery system as described herein does not include a conduit, but rather the detachment system is delivered directly to a target location.

### Delivery System

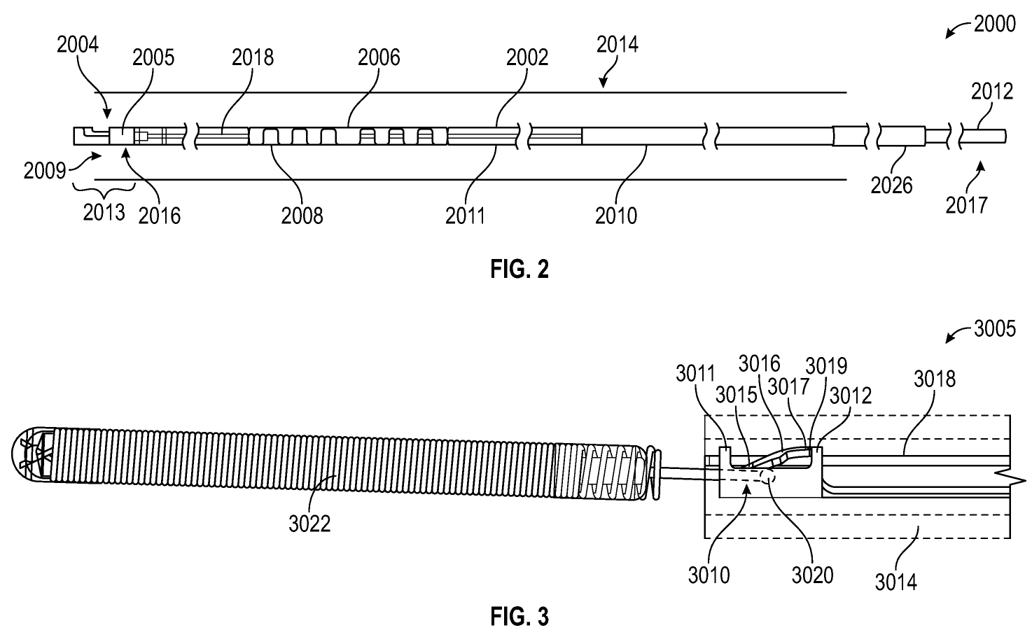

FIG. 2 shows an illustration of an exemplary embodiment of a delivery system 2000 for delivering and deploying an intravascular implant (not shown) such as an embolic coil. Other non-limiting examples of implants suitable for use with the systems, devices, and methods described herein include, for example, occluding coils and intravascular stents.

The delivery system 2000 comprises a conduit such as a standard catheter 2014 or micro-catheter (or other conduit) and a detachment system 2004. The detachment system 2004 comprises an elongate body that is configured to be slideably positioned (i.e., advanced and withdrawn) within the catheter 2014 and, in some embodiments, the detachment system 2004 is delivered via the catheter 2014 to a target location such as, for example, an intracranial aneurysm. That is, in embodiments of the delivery system 2000 that include the catheter 2014, the catheter 2014 with the detachment system 2004 within the catheter 2014 is typically delivered to a target location by a user, wherein a target location may comprise, for example, an intracranial aneurysm or, for example, an atherosclerotic lesion. In some embodiments, the detachment system 2004 may be delivered directly to a target location without the catheter 2014.

### Detachment System

The detachment system 2004 comprises a proximal end 2017 and a distal end 2016, which are each configured to include different functional elements of the detachment system 2004. In general, the proximal end 2017 of the detachment system 2004 remains outside of the patient during the use of the delivery system 2000, and the proximal end 2017 of the detachment system 2004 generally includes features that allow a user to manually direct the detachment system 2004 and control the deployment of an implant, such as an embolic coil. The proximal end 2017 of the detachment system 2004 is configured to provide a mechanism for manually deploying an implant at a target location by the user of the delivery system 2000. In general, the distal end 2016 includes a detachment mechanism 2005 that is configured to release or deploy an intravascular implant at a target location and a radiopaque marker 2006 that is positioned to align with a radiopaque marker on the distal end of the catheter 2014 when the detachment system 2004 is within a proper position relative to the catheter 2014 for implant deployment.

The detachment mechanism 2005 is disposed at a distal end 2016 of the detachment system 2004. In some embodiments of the delivery system 2000, the detachment system 2004 comprises an elongate body including a series of respectively optional interconnected tubes comprising an optional shrink tube 2002, an optional connecting tube 2010, an optional expansion tube 2026, and an optional grip tube 2012. The optional interconnected tubes 2002, 2010, 2026, and 2012 are each respectively configured to provide different qualities or features to the detachment system 2004.

The shrink tube 2002 comprises a flexible material such as a polymer, and is configured to cover and/or surround at least a portion of the distal end 2016 of the delivery system 2000 while providing flexibility to maneuver through bends in the vasculature system. The shrink tube 2002 also maintains a protective covering over the detachment mechanism 2005 to encapsulate a primary member 2018, discussed in more detail below. The shrink tube 2002 also maintains a tight coupling between the radiopaque marker 2006 and the detachment mechanism 2005 via mechanical coupling between the radiopaque marker 2006 and a radiopaque marker coupler 2008.

The connecting tube 2010 may optionally be connected to the shrink tube 2002 and comprises a relatively rigid material (as compared to the shrink tube 2002) that provides rigidity to portions of the distal end 2016 and/or proximal end 2017 so that the detachment system 2004 is more easily advanced and withdrawn within the catheter 2014.

The expansion tube 2026 is optionally connected to the connecting tube 2010 and provides a segment with an expanded diameter (as compared to the optional shrink tube 2002 and connecting tube 2010), providing ease of handling relative to the relatively small diameter optional shrink tube 2002 and connecting tube 2010. In some embodiments of the detachment system 2004, the expansion tube 2026 facilitates deployment of an implant from the detachment mechanism 2005 by facilitating manual withdrawal of the primary member 2018. A connecting wire 2011 connects the distal portion of the detachment system 2004.

In some embodiments of the delivery system 2000, the detachment mechanism 2005 is formed entirely from a memory metal material. In some embodiments, the memory metal material of the detachment mechanism 2005 is nitinol. In some embodiments of the detachment mechanism 2005, the detachment mechanism 2005, not including the primary member 2018 (which comprises a different material), comprises a memory material such as nitinol. In some embodiments of the detachment mechanism 2005, the detachment mechanism 2005, not including the radiopaque marker 2006 (which comprises a different material), comprises a memory material such as nitinol. In some embodiments of the detachment mechanism 2005, the detachment mechanism 2005, not including the primary member 2018 and the radiopaque marker 2006 (which comprise a different material), comprises a memory material such as nitinol.

The distal end 2016 of the detachment system 2004 includes the radiopaque marker 2006, and the distal end of the catheter 2014 includes a radiopaque marker (not shown). Non-limiting examples of metals suitable for use as either the radiopaque marker 2006 of the detachment system 2004 or the radiopaque marker on the catheter 2014 include noble metals or alloys such as platinum, platinum-tungsten, platinum iridium, silver, or gold. In some embodiments of the delivery system 2000, the radiopaque marker 2006 of the detachment system 2004 and the radiopaque marker on the catheter 2014 are positioned so that they align with one another when the detachment mechanism 2005 is positioned at a deployment location 2013. In some embodiments of the delivery system 2000, the radiopaque marker 2006 of the detachment system 2004 and the radiopaque marker of the catheter 2014 are positioned so that they align at a location about 30 mm proximal to the deployment location 2013.

In some embodiments of the delivery system 2000, the detachment system 2004 includes the radiopaque marker coupler 2008 at a radiopaque marker location. The radiopaque marker coupler 2008 is a portion of the detachment system 2004 that is configured to couple with the radiopaque marker 2006. That is, the radiopaque marker coupler 2008 of the detachment system 2004 typically comprises a metal such as, for example, platinum, platinum-tungsten, platinum iridium, silver, or gold. Because the remaining portions of the detachment mechanism 2005 (except in some embodiments the primary member 2018 is not present) comprise a memory material such as nitinol, coupling the radiopaque marker coupler 2008 to the detachment mechanism 2005 is not easily achievable with typical methods such as welding due to differences between the materials (i.e., between the radiopaque marker coupler 2008 and the memory material of the detachment mechanism 2005). As such, the radiopaque marker coupler 2008 is configured to couple with the radiopaque marker 2006 mechanically without the need for the two elements to be welded or similarly fused. In some embodiments of the detachment system 2004, the radiopaque marker coupler 2008 and the radiopaque marker 2006 have complimentary shapes that are configured so that the two components couple together by fitting together as shown in FIG. 2. That is, in some embodiments of the detachment system 2004, the radiopaque marker coupler 2008 is a component of the detachment mechanism 2005 that has an alternating tooth pattern (as shown) with elevations and indentations or, alternatively, for example, a sawtooth pattern, and likewise the radiopaque marker 2006 has a complimentary alternating tooth pattern (as shown) with elevations and indentations or, alternatively, for example, a sawtooth pattern so that the two components—the radiopaque marker coupler 2008 and the radiopaque marker 2006—fit together wherein an elevation of one component fits an indentation of the complementary component. In some embodiments of the detachment system 2004, the flexible shrink tube 2002 tightly surrounds these two coupled components—the radiopaque marker coupler 2008 and the radiopaque marker 2006—so that they are fixedly coupled together.

In order for the detachment system 2004 to properly deploy an implant such as an embolic coil within an aneurysm (i.e., the target), the detachment mechanism 2005 must be advanced to the deployment location 2013 along the catheter 2014. The deployment location 2013 may be a different location along the catheter 2014 depending on the type of implant deployed. For example, in some embodiments of the delivery system 2000, for proper deployment of an embolic coil or other implant within an aneurysm or other target location, the embolic coil or other implant is advanced entirely out of an aperture 2009 of the catheter 2014. For example, in some embodiments of the delivery system 2000, for proper deployment of an embolic coil or other implant within an aneurysm or other target location, the embolic coil is advanced partially out of the aperture 2009 of the catheter 2014. For example, in some embodiments of the delivery system 2000, for proper deployment of an embolic coil or other implant within an aneurysm or other target location, the detachment mechanism 2005 is advanced entirely out of the aperture 2009 of the catheter 2014. In some embodiments of the delivery system 2000, for proper deployment of an embolic coil or other implant within an aneurysm or other target location, the detachment mechanism 2005 is advanced partially out of the aperture 2009 of the catheter 2014. As shown in the exemplary embodiment shown in FIG. 2, a detachment mechanism 2005 in the illustrated embodiment is positioned within the distal portion of the catheter 2014 for proper deployment of an implant and as such the deployment location 2013 in the embodiment shown in FIG. 2 is located at the distal end of the catheter 2014.

That is, as shown in FIG. 2, the distal end 2016 of the catheter 2014 defines the deployment location 2013, which is a position or zone to where the detachment system 2004 (and thus the detachment mechanism 2005 at the distal end 2016 of the detachment system 2004) must be advanced in order to achieve successful deployment of an implant. For example, in embodiments of the delivery system 2000 wherein the detachment system 2004 remains entirely within the distal end of the catheter 2014 in order to achieve proper deployment of an implant, the deployment location 2013 is located where the detachment system 2004 is positioned within the distal end 2016 of the catheter 2014. For example, in embodiments of the delivery system 2000 wherein the detachment system 2004 is partially out of the aperture 2009 at the distal end 2016 of the catheter 2014 and partially within the distal end 2016 of the catheter 2014 in order to achieve proper deployment of an implant, the deployment location 2013 is located partially outside of the aperture 2009 and partially within the catheter 2014 where the detachment system 2004 is positioned. For example, in embodiments of the delivery system 2000 wherein the detachment system 2004 is completely out of the aperture 2009 in order to achieve proper deployment of an implant, the deployment location 2013 is located where the detachment system 2004 is positioned outside of the catheter 2014.

In general, the proximal end 2017 of the catheter 2014 is coupled to one or more features that provide a user with manual control over the advance of the implant to the target and deployment of the implant at or in the target. In some embodiments of the delivery system 2000, the proximal end 2017 of the catheter 2014 is coupled with the expansion tube 2026. The expansion tube 2026 is configured to have a larger diameter than the relatively small diameter of the shrink tube 2002. The expansion tube 2026 is generally configured so that it may couple the delivery system 2000 to other elements. For example, in some embodiments of the delivery system 2000, the expansion tube 2026 couples to the grip tube 2012 at the most proximal end 2017 of the delivery system 2000. The grip tube 2012 provides a user with a manual grip to advance and/or withdraw the detachment system 2004 in order to guide the detachment system through the vasculature of a patient. In some embodiments of the delivery system 2000, the expansion tube 2026 includes one or more oblong cuts or breaks within its material in order to facilitate a manual fracturing of the expansion tube 2026 so that the expansion tube 2026 is divided. Manually dividing the expansion tube 2026 provides a mechanism for withdrawing the fractured portion of the expansion tube 2026 away from the detachment system in a proximal direction, which is used in some embodiments of the delivery system 2000 to manually trigger deployment of an implant. In some embodiments of the delivery system 2000, the expansion tube 2026 couples with an external detachment device that is configured to manually trigger deployment of an implant using the detachment system 2004

### Detachment Mechanism

In some embodiments of the systems, devices, and methods described herein, the detachment mechanism 2005 is positioned at the distal end 2016 of the detachment system 2004, and the detachment system 2004 along with the catheter 2014 form the delivery system 2000.

In some embodiments, the detachment mechanism 2005 comprises a housing with a tab and a primary member 2018 that are configured to engage with an anchoring member of the intravascular implant. The user may position to actuate the detachment mechanism 2005, causing the detachment mechanism 2005 to release the anchoring member and deploy the intravascular implant.

The primary member 2018 as shown in FIG. 2 is sized to be long enough to extend the length of the delivery system 2000 when the detachment mechanism 2005 is at the deployment location 2013.

A number of additional features of embodiments of the detachment mechanism 2005 are now described with additional reference to FIG. 3 as well as further reference to FIG. 2.

FIGS. 3-5 show an illustration of an exemplary embodiment of a detachment mechanism 3005 that is detachably coupled to an embolic coil 3022 (or another implant). The embolic coil 3022 is coupled to the detachment mechanism 3005 and is disposed distal to the detachment mechanism 3005 in the catheter 3014. When the embolic coil 3022 is disposed within the catheter 3014, the embolic coil 3022 is in a linear configuration and the configuration of the coil 3022 may change when the coil 3022 is placed in the desired location. The embolic coil is configured to be slideably positioned (i.e., advanced and withdrawn) within the catheter 3014 along with the elongate body of detachment system 2004. The embolic coil 3022 may include an anchoring element 3020 that is detachably coupled to the detachment mechanism 3005. The anchoring element 3020 may be integral with the embolic coil 3022 or be coupled to the embolic coil 3022 at a proximal end of the embolic coil 3022.

The detachment mechanism 3005 comprises elements that are configured to cause the deployment of the embolic coil 3022. In some embodiments, the detachment mechanism 3005 includes a housing 3010 with a tab 3016 and a primary member 3018 that are engageable with the anchoring element 3020 of the embolic coil 3022.

In some embodiments, the housing 3010 with a distal end 3011 and a proximal end 3012. The housing 3010 defines the tab 3016 in a body of the housing 3010. The tab 3016 may have a proximal end 3017 that is coupled to or integral with a proximal end 3012 of the housing 3010 and a free end 3015. The free end 3015 of the tab 3016 is not coupled to the housing 3010 and extends toward the distal end 3011 of the housing 3010. The connection between the proximal end 3017 of the tab 3016 and the housing 3010 may form a hinge 3019 that the tab 3016 may rotate about. In other words, the hinge 3019 acts as an axis of rotation to enable the tab 3016 to rotate.

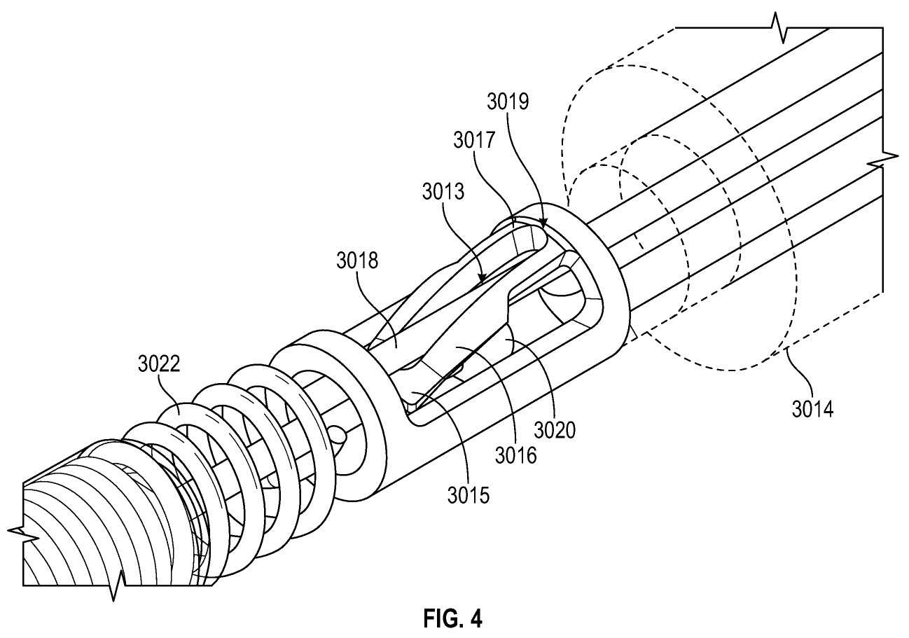

In some embodiments, the tab 3016 defines a substantially rectangular shape in which three edges of the tab 3016 are separated from the housing 3010 and the proximal end 3017 couples to the housing 3010. In other words, there is a gap between the tab 3016 and the housing 3010 on all three edges of the tab 3016 except the proximal end 3017 of the tab 3016. In some embodiments, the tab 3016 further includes an aperture 3013 as seen in FIG. 4. The aperture 3013 may comprise a substantially rectangular shape and have a width that is slightly greater than a width of the primary member 3018. The aperture 3013 is configured to receive the primary member 3018 so that the primary member 3018 may extend through the aperture 3013. In the illustrated embodiment, a distal end of the primary member 3018 may extend beyond the distal end 3011 housing 3010 of the detachment mechanism 3005 such that the distal end of the primary member is disposed outside the housing 3010.

In some embodiments, the tab 3016 is configured to have at least two configurations or positions: a first configuration or position and a second configuration or position. FIGS. 3 and 4 illustrate the tab 3016 in the first configuration or position. In the first configuration, the free end 3015 of the tab 3016 is deflected towards an interior of the housing 3010. The first configuration may be achieved when the primary member 3018 extends through the aperture 3013 to deflect the free end 3015 towards an interior of the housing 3010. When the tab 3016 is deflected towards the interior of the housing 3010 in its first position, it is positioned to couple directly with either the embolic coil 3022 that is at least partially within the housing 3010 or, in some embodiments, couple indirectly with the embolic coil 3022 by coupling with an anchoring element 3020 of the embolic coil 3022 that in some embodiments is coupled with the embolic coil 3022. When the primary member 3018 is engaged with the tab 3016, the tab 3016 may be deflected along the hinge 3019 and extend into the interior of the housing 3010. The hinge 3019 is perpendicular with an axis of the housing 3010 and the catheter 3014. By virtue of coupling to the tab 3016 in its first position, the embolic coil 3022 is held within the housing 3010 of the detachment mechanism 3005.

In some embodiments of the detachment mechanism 3005, the embolic coil 3022 (or other intravascular implant) is coupled with the anchoring element 3020 that is configured to couple with the tab 3016. In these embodiments, the anchoring element 3020 is configured to releasably couple with the tab 3016 when the tab 3016 is in the first position and deflected towards the interior of the housing 3010. For example, in some embodiments of the delivery system 3000, as shown in FIG. 3, the anchoring element 3020 comprises a sphere or ball and when the tab 3016 is in its first position, it hooks or latches the ball so that the ball is held within the housing 3010 and thus the embolic coil 3022 is held by the detachment mechanism 3005. In some embodiments, the ball is spherical and solid. As discussed above, in the first configuration, the primary member 3018 may extend through the aperture 3013 of the tab 3016 to deflect the tab 3016 toward the interior of the housing 3010. The primary member 3018 may further extend beyond the distal end 3011 of the housing 3010.

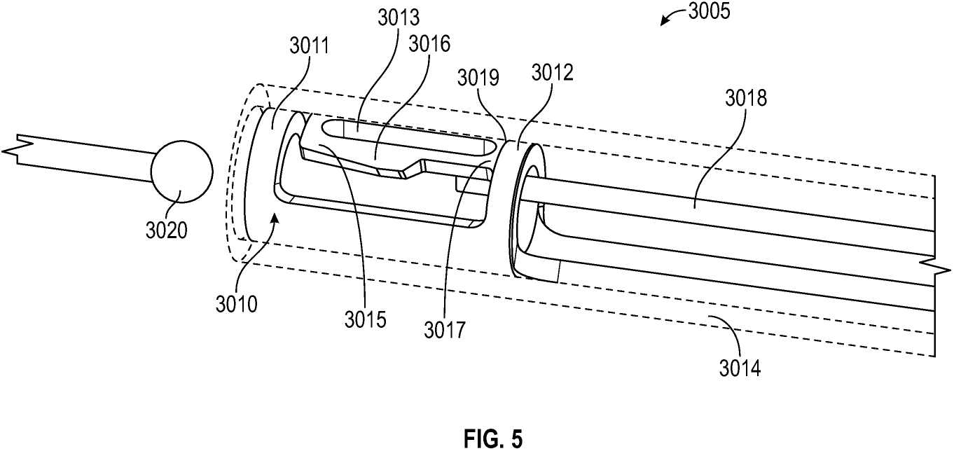

In embodiments of the detachment mechanism 3005 that include the primary member 3018, when the primary member 3018 is coupled with the tab 3016 it holds the tab 3016 in the first position of the tab 3016 so that the tab 3016 is deflected towards the interior of the housing 3010. When the primary member 3018, in these embodiments, is decoupled from the tab 3016 by being, for example, withdrawn in a proximal direction, the tab 3016 moves away from the interior of the housing 3010 to move to a second position as shown in FIG. 5. In embodiments where the tab 3016 comprises a memory material, the material of the tab 3016 facilitates its movement away from the interior of the housing 3010 when decoupled from the primary member 3018. In some embodiments of the delivery system 3000, the primary member 3018 is withdrawn proximally by a user when the detachment mechanism 3005 is positioned near a target location such as, for example, an intracranial aneurysm. The primary member 3018 may, for example, comprise a wire that extends out of the proximal end 3017 of the catheter 3014 to a location where the wire may be pulled proximally by a user thus decoupling the primary member 3018 and the tab 3016. The ability of the primary member 3018 to extend beyond the aperture 3013 of the tab 3016 provides some leeway so that slight proximal movement of the primary member 3018 does not dislodge the primary member 3018 from the aperture 3013. In other words, slight proximal movement of the primary member 3018 does not transition the tab 3016 from the first configuration. Instead, only a deliberate proximal pull on the primary member 3018 would dislodge the primary members from the tab 3016 to transition the tab 3016 from the first configuration to deploy the embolic coil 3022.

In some embodiments of the detachment system 3004, an expansion tube 3026 (not shown but similar to 2026) is configured to fracture so that at least a portion of the expansion tube 3026 may be withdrawn in a proximal direction away from the rest of the detachment system 3004. In some of these embodiments, the primary member 3018 is coupled with the expansion tube 3026 so that when the expansion tube 3026 is fractured and withdrawn in a proximal direction, the primary member 3018 is decoupled from the tab 3016 so that the tab 3016 moves from the first position to the second position and causes the release of the embolic coil 3022. In some embodiments of the delivery system 3000, the primary member 3018 comprises a wire that spans the length of the detachment system 3004 and extends out through the hole of the tab, 3016.

FIG. 5 shows an illustration of an embodiment of a distal end of the detachment mechanism 3005 showing how the anchoring element 3020 of the embolic coil 3022 is deployed from the detachment mechanism 3005. As described with reference to FIGS. 3 and 4, the tab 3016 has at least a first position and a second position. In the second position of the tab 3016, the tab 3016, as shown in FIG. 5, is positioned so that it is not deflected towards the interior of the housing 3010 but rather is positioned away from the interior of the housing 3010. The primary member 3018 is shown being withdrawn away from and thus decoupled from the tab 3016. As such, the anchoring element 3020 is no longer held by the detachment mechanism 3005 and the embolic coil 3022 (not shown in FIG. 5) is released or deployed at a target location.

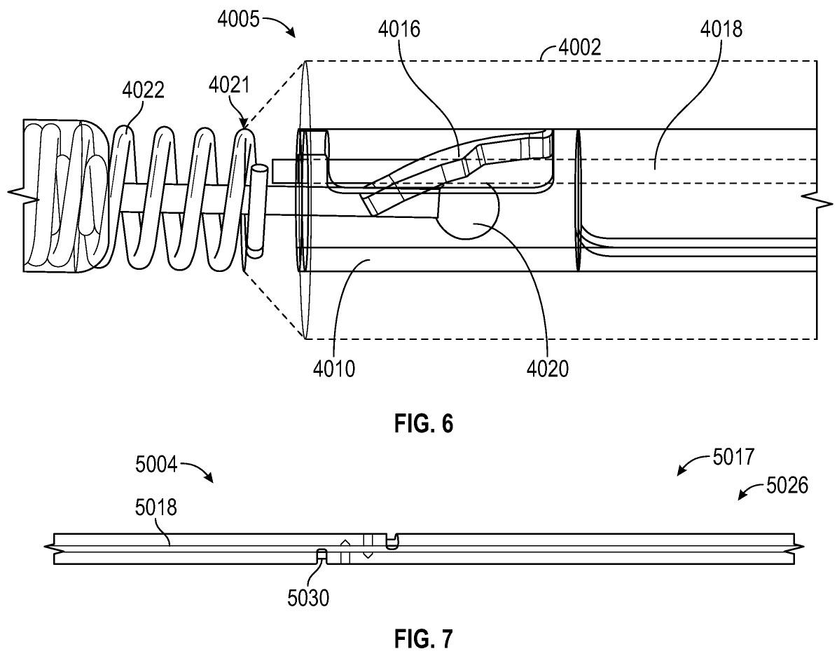

FIG. 6 illustrates another exemplary embodiment of a detachment mechanism 4005 that is detachably coupled to an embolic coil 4022 via an anchoring mechanism 4020. As discussed above, a tab 4016 in a first configuration secures the anchoring mechanism 4020 to prevent deployment of the embolic coil 4022 until a primary member 4018 is removed from the tab 4016 enabling the tab 4016 to go to a second position to release the embolic coil 4022. A covering 4002 encompasses a housing 4010 of the detachment mechanism 4005, a proximal end 4021 of the embolic coil 4022, and a distal end 4019 of the primary member 4018. While the covering encompasses the housing 4010 of the detachment mechanism 4005, a proximal end 4021 of the embolic coil 4022, and a distal end 4019 of the primary member 4018, for ease of illustration, the covering 4022 is shown in broken lines so that the underlining components may be seen. The covering 4002 may be a polymer that is heat shrunk to seal the detachment mechanism 4005 from bodily fluid during the implantation process.

### Expansion Tube

FIG. 7 shows an exemplary illustration of an embodiment of an expansion tube 5026. As described with reference to FIGS. 1 and 2, some embodiments of a detachment system 5004 include an expansion tube 5026 at a proximal end 5017 of the detachment system 5004. In some of these embodiments, the expansion tube 5026 includes one or more cuts 5030 at least partially surrounding the diameter of the expansion tube 5026 so that the cuts 5030 are positioned and/or configured to facilitate a fracture of the expansion tube 5026 when a bend is applied to the expansion tube 5026 by a user. Also shown in FIG. 5 is a primary member 5018 within the expansion tube 5026. As described with reference to FIGS. 1-2, a primary member 5018, in some embodiments of the detachment system 5004, is connected to the expansion tube 5026. When the expansion tube 5026 is fractured, the fractured portion of the expansion tube 5026 is able to be withdrawn away from the catheter in a proximal direction. In some embodiments of the detachment system 5004, when the expansion tube 5026 is fractured so that detachment system 5004 separates into a distal piece and a proximal piece that are able to be withdrawn from one another, the primary member 5018 is no longer held against the tab , so that the tab moves to a second position (facilitated by the memory material), which exerts a force on the primary member 5018 driving it proximally.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

What is claimed is:

1. An embolic coil delivery system for delivering and deploying an embolic coil at an aneurysm comprising:
an embolic coil with an anchoring element disposed at a proximal end of the embolic coil;
a conduit having a deployment location from which the embolic coil is deployed;
a detachment system configured to fit within the conduit and to be slideably advanced and withdrawn within the conduit, the detachment system comprising:
a housing that defines a tab with a first end coupled to a proximal end of the housing and a free end that extends toward a distal end of the housing, wherein the tab is configured to move from a first position to a second position, wherein when the tab is in the first position, the free end of the tab is deflected toward an interior of the housing; and
a primary member configured to engage and extend through the tab of the housing so that the tab is in the first position when engaged with the primary member and the tab is in the second position when the primary member is not engaged with the tab,
wherein the anchoring element of the embolic coil is configured to engage with the tab in the first position so that the embolic coil is coupled to the detachment system when the tab is in the first position, and wherein the anchoring element is configured to not engage with the tab in the second position so that the embolic coil is deployed when the tab is in the second position.

2. The embolic coil delivery system of claim 1, wherein the tab is integral with the housing.

3. The embolic coil delivery system of claim 1, wherein the tab rotates about a hinge.

4. The embolic coil delivery system of claim 3, wherein the hinge is perpendicular with an axis of the housing.

5. The embolic coil delivery system of claim 1, wherein the tab defines a rectangular shape, wherein three edges of the tab are separated from the housing and the tab defines a passthrough clearance aperture.

6. The embolic coil delivery system of claim 1, wherein the housing defines an aperture between the housing and the free end of the tab, the free end of the tab is opposite a hinge of the tab.

7. The embolic coil delivery system of claim 1, wherein the anchoring element is spherical and solid.

8. The embolic coil delivery system of claim 1, further comprising a covering that encompasses the entire housing and with the tab in the first position and a distal end of the primary member.

9. The embolic coil delivery system of claim 8, wherein the covering further encompasses a proximal portion of the embolic coil.

10. The embolic coil delivery system of claim 1, wherein the primary member extends beyond a distal end of the housing.

11. An embolic coil deployment system comprising:
an embolic coil with an anchoring element disposed at a proximal end, wherein the embolic coil is releasably coupled to a detachment mechanism via the anchoring element, the detachment mechanism comprising:
a housing that defines a tab, wherein the tab is configured to move from a first position to a second position, wherein when the tab is in the first position the tab is deflected toward an interior of the housing and configured to engage with the anchoring element of the embolic coil to couple the embolic coil to the detachment mechanism, wherein the tab defines a rectangular shape, wherein three edges of the tab are separated from the housing, and wherein the tab defines a passthrough clearance aperture.

12. The embolic coil deployment system of claim 11, wherein when the tab is in the second position, the tab is configured to not engage with the anchoring element of the embolic coil and to decouple the embolic coil from the detachment mechanism.

13. The embolic coil deployment system of claim 11, wherein the tab is disposed between a proximal end and a distal end of the housing.

14. The embolic coil deployment system of claim 11, wherein the tab rotates about a hinge.

15. The embolic coil deployment system of claim 14, wherein the hinge is perpendicular with an axis of the housing.

16. The embolic coil deployment system of claim 11, wherein the system further comprises a primary member configured to extend through the passthrough clearance aperture of the tab of the housing and engages with the tab of the housing so that the tab is in the first position when engaged with the primary member and the tab is in the second position when the primary member is not engaged with the tab.

17. The embolic coil deployment system of claim 11, wherein when the tab is in the first position and directed toward the interior of the housing a free end of the tab is disposed within the interior of the housing.
